(19) United States
(12) Patent Application Publication
(10) Pub. No.: US 2010/0124782 A1
(43) Pub. Date: May 20, 2010

(54) Composition For Repairing Defect In Skin Or Gingival Soft Tissue And Method Of Culturing Autologous Fibroblasts

(75) Inventor: Yasuyuki Ishizuka, Kanagawa (JP)

Correspondence Address: LAWRENCE Y.D. HO & ASSOCIATES PTE LTD, 30 BIDEFORD ROAD, #02-02, THONGSIA BUILDING, SINGAPORE, 229922, SG

(73) Assignee: Applied Cell Biotechnologies, Inc., Yokohama-shi, Kanagawa (JP)

(21) Appl. No.: 12/597,915

(22) PCT Filed: Apr. 25, 2008
(86) PCT No.: PCT/JP2008/001088

§ 371 Date: Oct. 28, 2009

(30) Foreign Application Priority Data
Apr. 28, 2007 (JP) 2007-120371

(51) Int. Cl. C12N 5/077

(52) U.S. Cl. 435/394; 435/325

(57) ABSTRACT

The object of the present invention is to provide a method for producing aggregates of autologous fibroblasts for producing autologous fibroblasts having been aggregating in a fibrous state, and an aggregate produced by this method. Another object is to provide a composition for repairing a defect in a skin or gingival soft tissue which can keep favorable treatment effect by remaining in the treated area over a long period of time, and an injection having this composition.

The above-mentioned problems are solved by a method of producing an aggregate of autologous fibroblasts which comprises the step of culturing autologous fibroblasts in a culture medium with vitamin C added and the step of culturing autologous fibroblasts by a culture method selected from among rotary culture, shaking culture and slant culture so as to form a fibrous aggregate by an extracellular matrix containing collagen secreted by the autologous fibroblasts, the aggregate produced in this way, and so on.

### TECHNICAL FIELD

The present invention relates to, for example, a composition for repairing a defect in a skin or gingival soft tissue, wherein autologous fibroblasts can be kept in the treated area by using an aggregate of autologous fibroblasts covered by an extracellular matrix containing autologous collagen.

### BACKGROUND ART

In order to repair a defect in soft tissue of the skin, methods of injecting collagen, autologous fibroblasts, etc. into a face etc. are known. For example, Japanese Patent Publication No. H7-10763 (see Patent Document 1 below) discloses a cosmetic surgery agent containing collagen.

Furthermore, Japanese Patent No. 3559566 (see Patent Document 2 below) discloses suspended matter with autologous passage dermal fibroblasts suspended in a carrier solution. On the other hand, in this official gazette, discloses a method of attracting in a syringe an aggregate obtained by incubating autologous dermal fibroblasts in a serum-free medium and administering it to a patient (see p. 6, 11.3-18 in the official gazette, for example).

However, there is a big medical problem for the technology disclosed in the above official gazette. That is, although the method uses a small amount of plasma of a subject, it also uses a culture solution using plasma of the same species (other people), fibrin or non-human serum, and thus there is a risk of infection by a virus etc. Furthermore, in the above official gazette, cells are not combined with one another, but are sprinkled on an aggregate of plasma. Moreover, even if autologous fibroblasts and an aggregate of plasma are injected together into the local area, fibroblasts move rapidly within the living body as fibroblasts have no scaffolds. As a result, fibroblasts do not remain in the desired treated area, resulting in a problem of ineffective treatment. Also, the official gazette is aimed at obtaining suspended matter, not a cellular aggregate.

Furthermore, Japanese Patent Publication No. 2005-532090 (see Patent Document 3 below) discloses an invention on the treatment using autologous fibroblasts. The official gazette discloses a composition containing autologous passage fibroblasts and muscle cells which are embedded in a matrix containing collagen (see claim 8 and paragraphs [0064] through [0067], for example).

On the other hand, Japanese Patent Publication No. 2005-532090 relates to the treatment of urinary incontinence, a vesicoureteral reflex, or a gastroesophageal reflux phenomenon. Also, the invention disclosed in the official gazette, as described in claim 1, relates to “suspended matter of autologous passage dermal fibroblasts containing substantially no protein derived from culture-medium serum and cells other than the fibroblasts”. Therefore, the invention described in the official gazette does not administer autologous fibroblasts covered with an extracellular matrix containing collagen. Similarly, the invention disclosed in the official gazette does not use autologous collagen to repair a defect in soft tissue of the skin.

Furthermore, Japanese Patent Publication No. 2006-510399 (see Patent Document 4 below) disclose an invention on the treatment for repairing a defect in a skin, soft tissue, and bone. The official gazette discloses a composition for repairing tissue containing undifferentiated mesenchymal cells and fibroblasts. However, as undifferentiated cells have a short cell cycle and tend to repeat division growth rapidly, there is a risk of canceration as well as a problem of poor prognosis.

[Patent Document 1] Japanese Patent Publication No. H7-10763

[Patent Document 2] Japanese Patent No. 3559566

[Patent Document 3] Japanese Patent Publication No. 2005-532090

[Patent Document 4] Japanese Patent Publication No. 2006-510399

### DISCLOSURE OF THE INVENTION

### Problems to be Solved by the Invention

It is an object of the present invention to provide a method of producing an aggregate of autologous fibroblasts for producing autologous fibroblasts which have been aggregating in a fibrous state, and an aggregate produced in this way.

It is an object to provide a composition for repairing a defect in a skin or gingival soft tissue which can keep favorable treatment effect by remaining in the treated area over a long period of time, and an injectable solution having this composition.

### Means for Solving Problems

If an aggregate exists in an injection solution when a liquid medicine is administered by injection, the aggregate gets stuck inside an injection needle, which prevents smooth injection. For this reason, when fibroblasts, collagen, etc. are administered by injection, typically, an aggregation inhibitor is added to an injection solution so as not to form an aggregate, a lump which is composed of aggregated autologous fibroblasts. However, as autologous fibroblasts have no scaffolds even if they are injected into the local area, they move rapidly within the living body. As a result, fibroblasts do not remain in the desired treated area, resulting in a problem of ineffective treatment. Consequently, the present invention is basically based on knowledge that by producing intentionally an aggregate which is not considered to be suitable for injection and administering the aggregate, scaffolds by collagen can be provided, and as a result, autologous fibroblasts can remain in the affected area over a long period of time.

The first aspect of the present invention relates to a method of producing an aggregate of autologous fibroblasts. That is, it relates to a method of producing an aggregate of autologous fibroblasts comprising the step of culturing autologous fibroblasts in a culture medium with vitamin C added and the step of culturing autologous fibroblasts by a culture method selected from among rotary culture, shaking culture and slant culture so as to form a fibrous aggregate by an extracellular matrix containing collagen secreted by the autologous fibroblasts. As shown in the examples described below, a fibrous aggregate can be produced through these steps. Furthermore, as shown in the examples described below, the aggregate produced through the above steps contains a large amount of collagen. Therefore, a fibrous aggregate where fibroblasts are tightly bound to one another can be produced. Furthermore, as shown in the examples described below, an aggregate having protease resistance can be produced. That is, according to the production method of the present invention, an aggregate with high treatment effect can be manufactured which is not easy to move out of the affected area and is not easy to be dispersed by protease within the living body when it is administered to the affected area.

A preferred embodiment of the first aspect of the present invention is the production method described above further comprising the step of exfoliating cells with a separation liquid containing trypsin. As shown in the examples described below, in the production method of the present invention, the obtained fibrous aggregate contains a large amount of collagen, and it is not dispersed even if it is exfoliated or removed using a separation liquid containing trypsin. That is, it has protease resistance. Therefore, according to the production method of the present invention, an aggregate with high treatment effect can be produced which is not easy to move out of the affected area and is not easy to be dispersed by protease within the living body when it is administered to the affected area.

A preferred embodiment of the first aspect of the present invention is the production method described above, wherein the step of culturing in a culture medium is the step of culturing in an autologous serum medium or a serum-free medium. By using an autologous serum medium or a serum-free medium as a serum medium, an aggregate which causes fewer inflammatory responses in the affected area when it is administered to the affected area can be produced. Furthermore, by using self-derived material, infection by a virus etc., contamination, etc. can be prevented, resulting in high treatment effect.

A preferred embodiment of the first aspect of the present invention is the production method described above, wherein the autologous fibroblasts are either skin-derived fibroblasts or gingival-derived fibroblasts.

A preferred embodiment of the first aspect of the present invention is the production method described above, wherein the step of culturing is a step of culturing by rotary culture, the rotary culture carried out at a rate of 0.1-2 revolutions per minute (“A-B” means “between A and B, both inclusive”. The same shall apply hereinafter). As rotary culture can provide directivity for collagen secreted from fibroblasts according to the rotation direction, a fibrous aggregate can be obtained effectively.

A preferred embodiment of the first aspect of the present invention is the manufacturing method described above, wherein the step of culturing is a step of culturing by shaking culture, the shaking culture being any of vertical motion culture, horizontal motion culture, or gyratory culture, and the shaking culture carried out at a rate of 5-50 times per minute. As shown in the examples described below, shaking culture can provide directivity for collagen secreted from fibroblasts according to the shaking direction, and thus a fibrous aggregate can be obtained effectively.

A preferred embodiment of the first aspect of the present invention is the production method described above, wherein the step of culturing is a step of culturing by rotary culture, the step of culturing by rotary culture comprising the step of culturing cells at a rate of 0.1-0.5 revolutions per minute, the step of adding vitamin C to a culture medium so that it may be set to 0.01-1 mM and culturing cells at a rate of 1.5-2 revolutions per minute, and the step of culturing cells at a rate of 0.5-1.5 revolutions per minute in this order. By these culturing steps with changed rotation speed, a fibrous aggregate of fibroblasts with collagen combined with one another efficiently secreted from the fibroblasts can be obtained. Furthermore, as the method can provide directivity for collagen secreted from the fibroblasts, a fibrous cellular aggregate can be produced effectively.

A preferred embodiment of the first aspect of the present invention is the production method described above, wherein the step of culturing is a step of culturing by shaking culture, the step of culturing by shaking culture comprising the step of culturing cells at a rate of 5-10 times per minute, the step of adding vitamin C to a culture medium so that it may be set to 0.01-1 mM and culturing cells at a rate of 20-30 times per minute, and the step of culturing cells at a rate of 10-20 times per minute in this order. By these culturing steps with changed shaking times, a fibrous aggregate of fibroblasts with collagen combined with one another efficiently secreted from the fibroblasts can be obtained. Furthermore, as the method can provide directivity for collagen secreted from the fibroblasts, a fibrous cellular aggregate can be produced effectively.

A preferred embodiment of the first aspect of the present invention relates to an aggregate manufactured by the method described above. As shown in the embodiments described below, the aggregate manufactured in this way is one which contains a large amount of collagen and is in a fibrous state with autologous fibroblasts combined with one another through an extracellular matrix containing collagen secreted from the autologous fibroblasts. Furthermore, as shown in the embodiments described below, the aggregate of the present invention has protease resistance. The aggregate combined by an extracellular matrix containing collagen is not easy to move out of the affected area because the extracellular matrix serves as a scaffold when the aggregate is administered to the affected area. Moreover, the aggregate has protease resistance and thus is not easy to be dispersed by protease within the living body. Therefore, the aggregate of the present invention can remain in the affected area, resulting in high treatment effect. Furthermore, the present invention can administer a large number of cells together to the affected area, resulting in high growth ability of fibroblasts and high treatment effect.

A second aspect of the present invention relates to a composition for repairing a defect in soft tissue comprising an aggregate and a pharmaceutically acceptable liquid medicine. The aggregate is produced by a method of producing an aggregate of fibroblasts comprising the step of culturing in an autologous serum medium or a serum-free medium with vitamin C added, and the step of culturing the autologous fibroblasts by a culture method selected from among rotary culture, shaking culture and slant culture so as to form a fibrous aggregate by an extracellular matrix containing collagen secreted by the autologous fibroblasts. The pharmaceutically acceptable liquid medicine is a liquid medicine containing carbohydrates, electrolytes and amino acids. As described above, the aggregate contained in the composition of the present invention contains a large amount of collagen and has protease resistance. And protein of animals or other people is not used, resulting in the reduction of risks such as inflammatory responses or virus infection. As shown in the examples described below, the survivability of autologous fibroblasts can be improved when they are suspended in a liquid medicine (e.g. high calorie infusion solution) containing electrolytes and amino acids. Therefore, the composition of the present invention can provide high treatment effect and thus can be used preferably for repairing a defect in soft tissue.

A preferred embodiment of the second aspect of the present invention is a composition described above wherein the aggregate is an aggregate containing differentiated fibroblasts. As the composition contains differentiated fibroblasts, it can reduce the risk of canceration of fibroblasts after administration, and it can be used preferably as a composition for repairing a defect in soft tissue.

The third aspect of the present invention relates to a method of producing an injectable solution comprising the step of culturing in an autologous serum medium or a serum-free medium with vitamin C added, the step of culturing autologous fibroblasts by a culture method selected from among rotary culture, shaking culture and slant culture so as to form a fibrous aggregate by an extracellular matrix containing collagen secreted by the autologous fibroblasts, and the step of collecting the cultured cells as an aggregate, wherein after the aggregate obtained in the step of collecting is inserted into a syringe through a first injection needle, the first injection needle is removed from the syringe and a second injection needle with a smaller inner diameter than that of the first injection needle is attached to the syringe.

In this way, an aggregate is once sucked up with an injection needle with a larger inner diameter, which is later replaced by an injection needle with a smaller inner diameter. Consequently, the aggregate can be formed into an appropriate size during treatment, and physical damage to cells can be minimalized, and in addition the situation that an aggregate clots in an injection needle can be prevented effectively.

### Effect of the Invention

According to the present invention, a method of producing an aggregate of autologous fibroblasts for producing autologous fibroblasts which have been aggregating in a fibrous state, and an aggregate produced in this way can be provided.

According to the present invention, a composition for repairing a defect in a skin or gingival soft tissue which can keep favorable treatment effect by remaining in the treated area over a long period of time, and an injection having this composition can be provided.

### BRIEF DESCRIPTION OF THE DRAWINGS

FIG. 1 shows a photograph replacing a drawing which shows a liquid medicine containing fibroblasts obtained from Example 1.

FIG. 2 shows a photograph replacing a drawing which shows a liquid medicine containing fibroblasts obtained by a conventional method.

FIG. 3 shows a photograph replacing a drawing which shows a situation where the aggregate obtained from Example 1 is dispersed.

FIG. 4 shows a photograph replacing a drawing which shows a situation where cellular aggregates of fibroblasts are dispersed.

FIG. 5 shows a photograph replacing a drawing which shows a situation where cells are suspended in a high calorie injection solution.

FIG. 6 shows a photograph replacing a drawing which shows a situation where cells are suspended in a physiological salt solution.

FIG. 7 shows a photograph replacing a drawing which shows fibroblasts obtained by stationary culture or rotary culture. FIG. 7A shows fibroblasts obtained by stationary culture, and FIGS. 7B and 7C show fibroblasts obtained by rotary culture.

FIG. 8 shows a photograph replacing a drawing which shows cells which were kept at 4 degrees C. for a certain amount of time. FIG. 8A shows a photograph replacing a drawing which shows cells 24 hours after the start of culture. FIG. 8B shows a photograph replacing a drawing which shows cells 48 hours after the start of culture.

### BEST MODE FOR CARRYING OUT THE INVENTION

The first aspect of the present invention relates to a method of producing an aggregate of autologous fibroblasts. That is, it relates to a method of producing an aggregate of autologous fibroblasts comprising the step of culturing autologous fibroblasts in a autologous serum medium or a serum-free medium with vitamin C added and the step of culturing autologous fibroblasts by a culture method selected from among rotary culture, shaking culture and slant culture so as to form a fibrous aggregate by an extracellular matrix containing collagen secreted by the autologous fibroblasts. Conventionally, when fibroblasts were cultured, cellular aggregates of fibroblasts sometimes could be obtained by chance. However, such cellular aggregates were usually removed, and the cells were dispersed with one another and thus they were easy to move even if they were injected into the living body. On the other hand, the present invention aims to obtain an aggregate of autologous fibroblasts by actively causing the autologous fibroblasts to secrete an extracellular matrix containing collagen. In the present invention, as autologous fibroblasts are combined with one another through an extracellular matrix, an aggregate can be produced which has a good scaffold when injected into the living body and remains in the affected area for a long period of time.

Fibroblasts for producing an aggregate can be extracted from soft tissue etc. to be repaired and cultured by well-known methods. Culture conditions can be adjusted appropriately according to the origins of the fibroblasts to be cultured by a person skilled in the art. Furthermore, a person skilled in the art can appropriately use a well-known culture medium as a culture medium where fibroblasts are cultured according to the origins of the fibroblasts to be cultured. As the aggregate of the present invention is administered to the patient who provides fibroblasts for producing the aggregate, the culture medium where fibroblasts are cultured is preferably an autologous serum medium or a serum-free medium with no animal derived component (e.g. ASF culture medium 104). As protein of animals or other people is not used, the risks of virus infection, contamination, etc. can be reduced. Moreover, immune responses can be prevented which occur when protein of animals or other people is administered to a patient.

An “extracellular matrix containing collagen secreted by autologous fibroblasts” contains autologous collagen obtained by culturing autologous fibroblasts, fibronectin which has the function of combining collagen with one another, etc. As shown in the examples described below, a specific extracellular matrix includes type I collagen, type VI collagen, fibronectin, vimentin, microfibril-associated glycoprotein, etc. In addition, as the main component of an extracellular matrix is typically autologous collagen, “including an extracellular matrix containing autologous collagen” herein may be read as “including autologous collagen”.

In the culture steps of the present invention, 0.001-10 mM of vitamin C (preferably 0.01-1 mM, more preferably 0.05-0.5 mM]) is added to a culture medium in culturing autologous fibroblasts. And collagen is secreted from autologous fibroblasts and an aggregate of autologous fibroblasts is thereby obtained. The culture period for obtaining the aggregate of the present invention is from 2 days to 1 month, for example, preferably from 4 to 20 days, more preferably from 7 to 14 days. It is desirable to change culture media every several days in the culture steps. The period for changing culture media is from 1 to 7 days, for example, preferably from 2 to 4 days. By changing culture media periodically, the survivability of cells to be cultured for a long period of time can be improved, resulting in producing an aggregate with high treatment effect. Moreover, in changing culture media, the concentration of vitamin C added to culture media may be increased every time culture media are changed. By gradually increasing the concentration, the directivity of collagen can be lined up, resulting in producing a fibrous aggregate effectively.

In the present invention, the direction of collagen refers to the extension direction of collagen. Giving directivity to collagen refers to extending collagen in a specific direction. And lining up the direction of collagen refers to extending collagen secreted from a plurality of fibroblasts toward the same direction respectively.

In the culture steps of the present invention, autologous fibroblasts are cultured by a culture method selected from among rotary culture, shaking culture and slant culture so as to form a fibrous aggregate by an extracellular matrix containing collagen secreted by the autologous fibroblasts.

Rotary culture can be carried out using well-known vessels for rotary culture, such as a cylindrical culture vessel. The revolution speed in the case of carrying out rotary culture is 0.01-5 revolutions per minute, preferably 0.05-3 revolutions per minute, more preferably 0.1-2 revolutions per minute. As for the rotary culture steps, the revolution speed may be changed in the middle of the culture steps. Specific culture steps of changing the revolution speed includes the step of culturing at a rate of 0.1-0.5 revolutions per minute for 1 to 2 days after the start of rotary culture, the step of subsequently culturing at a rate of 1.5-2 revolutions per minute until the cells become 70-80% confluent, and the step of subsequently culturing at a rate of 0.5-1.5 revolutions per minute until the end of culture. The rate of the confluent of cells can be determined appropriately by a person skilled in the art. Though vitamin C may be added to culture media in the whole steps, vitamin C is preferably added to the culture media in the step of culturing at a rate of 1.5-2 revolutions per minute and the step of culturing at a rate of 0.5-1.5 revolutions per minute. The concentration of vitamin C added in these steps is 0.001-10 mM, for example, preferably 0.01-1 mM, more preferably 0.05-0.5 mM. Owing to these steps, the directivity of collagen can be lined up, resulting in producing a fibrous aggregate effectively.

Shaking culture includes vertical motion culture, horizontal motion culture, gyratory culture, etc. These cultures can be carried out using well-known vessels for culture having a flat culture surface. Well-known equipment can be used as shaking culture equipment for shaking culture.

Vertical motion culture is shaking culture carried out in a seesaw state. In vertical motion culture, if vertical motion culture equipment, for example, has a supporting point in the center of a plate whereon a culture vessel is mounted and both ends of the plate move up and down, one cycle is counted as one time when one end of the plate moves upward or downward from a horizontal state and moves in opposite direction back to a horizontal state. The shaking speed in the case of vertical motion culture is 1-50 times per minute, for example, preferably 5-30 times per minute, more preferably 10-20 times per minute. The angle where vertical motions in a seesaw state are carried out is ±1-15 degrees if the position where a culture vessel is placed horizontally is set to 0 degree, preferably ±3-13 degrees, more preferably ±5-10 degrees.

As for vertical motion culture steps, the shaking speed or shaking angle may be changed in the middle of the culture steps. Specific culture steps of changing the shaking speed includes the step of culturing at a rate of 5-10 times per minute for 1 to 2 days after the start of vertical motion culture, the step of subsequently culturing at a rate of 20-30 times per minute until the cells become 70-80% confluent, and the step of subsequently culturing at a rate of 10-20 times per minute until the end of culture. Specific culture steps of changing the shaking angle includes the step of culturing at an angle of ±1-2 degrees 1 to 2 days after the start of vertical motion culture, the step of subsequently culturing at an angle of ±10-13 degrees until the cells become 70-80% confluent, and the step of subsequently culturing at an angle of ±5-10 degrees until the end of culture. Though vitamin C may be added to culture media in the whole steps, vitamin C is preferably added to the culture media in the step of culturing at a rate of 20-30 times per minute and the step of culturing at a rate of 10-20 times per minute in the case of the steps of changing the shaking speed. Similarly, vitamin C is preferably added to the culture media in the step of culturing at an angle of ±10-13 degrees and the step of culturing at an angle of ±5-10 degrees in the case of the steps of changing the shaking angle. The concentration of vitamin C added in these steps is 0.001-10 mM, for example, preferably 0.01-1mM, more preferably 0.05-0.5 mM. As for culture steps of changing the shaking speed and shaking angle, either of the shaking speed or shaking angle may be unchanged, or both steps may be changed. Owing to these steps, the directivity of collagen can be lined up, resulting in producing a fibrous aggregate effectively.

Horizontal motion culture is shaking culture which goes and comes back in one direction while keeping the horizontal surface, and one round-trip is counted as one time. The shaking speed in the case of horizontal motion culture is 1-50 times per minute, for example, preferably 5-30 times per minute, more preferably 10-20 times per minute. Specific culture steps of changing the shaking speed includes the step of culturing at a rate of 5-10 times per minute for 1 to 2 days after the start of horizontal motion culture, the step of subsequently culturing at a rate of 20-30 times per minute until the cells become 70-80% confluent, and the step of subsequently culturing at a rate of 10-20 times per minute until the end of culture. Though vitamin C may be added to culture media in the whole steps, vitamin C is preferably added to the culture media in the step of culturing at a rate of 20-30 times per minute and the step of culturing at a rate of 10-20 times per minute in the case of the steps of changing the shaking speed. The concentration of vitamin C added in these steps is 0.001-10 mM, for example, preferably 0.01-1 mM, more preferably 0.05-0.5 mM. Owing to these steps, the directivity of collagen can be lined up, resulting in producing a fibrous aggregate effectively.

Gyratory culture is shaking culture which gyrates in a constant direction while keeping the horizontal surface. One time in gyratory culture refers to one revolution. The shaking speed in the case of gyratory culture is 1-50 times per minute, for example, preferably 5-30 times per minute, more preferably 10-20 times per minute. As for the gyratory culture steps, the revolution speed may be changed in the middle of the culture steps. Specific cultivation steps of changing the revolution speed include the step of culturing at a rate of 1-3 revolutions per minute for 1 to 2 days after the start of gyratory culture, the step of subsequently culturing at a rate of 20-30 revolutions per minute until the cells become 70-80% confluent, and the step of subsequently culturing at a rate of 10-20 revolutions per minute until the end of culture. Though vitamin C may be added to culture media in the whole steps, vitamin C is preferably added to the culture media in the step of culturing at a rate of 20-30 revolutions per minute and the step of culturing at a rate of 10-20 revolutions per minute. The concentration of vitamin C added in these steps is 0.001-10 mM, for example, preferably 0.01-1 mM, more preferably 0.05-0.5 mM. Owing to these steps, the directivity of collagen can be lined up, resulting in producing a fibrous aggregate effectively.

Slant culture can be carried out using well-known vessels for culture having a flat culture surface. The slant angle at the time of slant culture is 1-15 degrees, for example, preferably 5-10 degrees. It is preferable to carry out slant culture horizontally without slant for 1 to 2 days after the start of culture and to subsequently carry out slant culture at a slant angle of 1-15 degrees until the end of culture. Though vitamin C may be added to the culture media from the start of culture, vitamin C may be added after the start of slant culture. The concentration of vitamin C added is 0.001-10 mM, for example, preferably 0.01-1 mM, more preferably 0.05-0.5 mM. Owing to these steps, the directivity of collagen can be lined up, resulting in producing a fibrous aggregate effectively.

After the end of the cultivation steps of the present invention, cells can be exfoliated by a well-known method, and the aggregate of cells can be collected. A well-known separation liquid can be used as a separation liquid exfoliating the aggregate of cells of the present invention. A specific separation liquid contains trypsin or EDTA, for example. In order to obtain a fibrous aggregate from the cells by stationary culture, it is necessary to use a separation liquid of low resolution EDTA or a non-enzymatic separation liquid so that the cells may not be dispersed. However, as shown in the examples described below, the aggregate produced by the methods of the present invention does not get loose even if enzyme such as trypsin having protease activity is used because fibroblasts are strongly combined with one another by an extracellular matrix containing collagen secreted from autologous fibroblasts. Therefore, a separation liquid containing high resolution protease etc. such as trypsin may be used to exfoliate cells.

Preferably, the trypsin contained in a separation liquid used in the step of exfoliating cells of the present invention does not use animal derived material. By using non-animal-derived trypsin, immune responses to animal-derived trypsin can be prevented when the produced aggregate is administered to the affected area. Well-known trypsin such as trypsin which is commercially available recombinant enzyme may be used as non-animal-derived trypsin.

Skin-derived or gingival-derived fibroblasts are preferable as fibroblasts for producing the aggregate of the present invention. As skin-derived or gingival-derived fibroblasts have a large amount of collagen secretion, a fibrous aggregate can be produced effectively.

As shown in the examples described below, the aggregate of the present invention is an aggregate which contains a large amount of collagen and is in a fibrous state with autologous fibroblasts combined with one another through an extracellular matrix containing collagen secreted from the autologous fibroblasts. Furthermore, as shown in the examples described below, the aggregate of the present invention has protease resistance. This aggregate combined by an extracellular matrix containing collagen is not easy to move out of the affected area because the extracellular matrix serves as a scaffold when the aggregate is administered to the affected area. Moreover, the aggregate has protease resistance and thus it is not easy to be dispersed by protease within the living body. Furthermore, the aggregate produced using an autologous serum medium or a serum-free medium does not easily cause inflammatory responses in the affected area when the aggregate is administered to the affected area. Moreover, by using self-derived material, infection by virus etc., contamination, etc. can be prevented, resulting in high treatment effect. The aggregate produced by the production methods of the present invention does not use protein of animals or other people. Therefore, the aggregate produced by the production methods of the present invention can reduce the risks of infection by virus, contamination, etc. Therefore, the aggregate of the present invention can remain in the affected area and can reduce the risks of immune responses, virus infection etc. which occur when the aggregate is administered, resulting in high treatment effect. Thus, the aggregate can be used appropriately for repairing a defect in soft tissue.

The second aspect of the present invention relates to a composition for repairing a defect in soft tissue comprising an aggregate and a pharmaceutically acceptable liquid medicine. The aggregate is produced by a method of producing an aggregate of fibroblasts comprising the step of culturing in an autologous serum medium or a serum-free medium with vitamin C added, and the step of culturing the autologous fibroblasts by a culture method selected from among rotary culture, shaking culture and slant culture so as to form a fibrous aggregate by an extracellular matrix containing collagen secreted by the autologous fibroblasts. The pharmaceutically acceptable liquid medicine is a liquid medicine containing carbohydrates, electrolytes and amino acids. A well-known liquid medicines used in an injectable solution can be used appropriately as a pharmaceutically acceptable liquid solution. A specific liquid solution includes purified water, distilled water, physiological salt solution, etc. The composition of the present invention can be obtained by exfoliating an aggregate, separating cells by centrifugation, subsequently washing with cleaning fluid such as a physiological salt solution, and adding a pharmaceutically acceptable physiological salt solution. The composition of the present invention may contain dispersed cells which have not formed an aggregate. The dispersed cells can fill the gap between aggregates when the composition is administered to the affected area, resulting in improving treatment effect. In the composition of the present invention, the rate of aggregates to dispersed cells is 50:50-99:1, for example. It may be 70:30-80:20 or 80:20-95:5. If the rate of dispersed cells is too high, it may be more difficult to keep the cells in the affected area. Therefore, the rate is preferably 80:20-95:5.

On the other hand, as shown in the examples described below, the survivability of autologous fibroblasts can be improved when they are suspended in a liquid medicine such as a high calorie infusion solution containing sugars, electrolytes and amino acids. A well-known high calorie infusion solution may be used. Glucose is mainly used as a sugar. Fructose, xylitose, or sorbitose may be used as sugars other than glucose. Electrolytes include Na (sodium), K (potassium), Cl (crawl), Ca (calcium), Mg (magnesium), etc. Moreover, trace metals such as Zn (zinc), P (Lynn), Fe (iron), and Cu (copper), and organic acids such as citrate, gluconic acid, acetic acid (acetate), and lactic acid (lactate) may be added if desired. Amino acids include glycine, L-alanine, L-proline, L-aspartic acid, L-serine, L-tyrosine, L-glutamic acid, L-cysteine, L-leucine, L-isoleucine, L-valine, L-lysine,

L-methionine, and L-phenylalanine, L-threonine, L-tryptophan, L-arginine, or L-histidin. Compositions in well-known high calorie infusions may be adopted appropriately.

The composite of the present invention is preferably kept at low temperature (for example, 1-10 degrees C., preferably 2-8 degrees C.). The holding time is less than 5 days, preferably less than 12 hours, most preferably less than 4 hours. As shown in the examples described below, even after the aggregate of the present invention is preserve at low temperature for a certain period of time, the growth ability of the cells is maintained. Therefore, by administering the composition containing such an aggregate to the affected area, a high treatment effect can be obtained. Although the composite of the present invention, which was preserved at low temperature, may be administered to the affected area at low temperature, it is preferable to administer the composition after warming it so that its temperature may be 34-37 degrees C. By warming a composition before administration, cell growth after administration to the affected area or adhesiveness of the aggregate to the affected area can be improved.

The preferable embodiment of the second aspect of the present invention is a composition comprising an aggregate containing differentiated fibroblasts. A well-known culture method may be used to differentiate fibroblasts. Though the differentiation speed of cells depends on culture length and/or culture conditions, it can be adjusted appropriately by a person skilled in the art. The degree of differentiation of cells can be measured by a well-known method. For example, the activity of protein which develops only in undifferentiated cells. Moreover, protein which develops only in undifferentiated cells may be dyed. The degree of differentiation of cells can be measured appropriately by a person skilled in the art. As undifferentiated cells have a shorter cell cycle and tend to repeat mitotic growth actively, they have a risk of canceration compared to differentiated cells. On the other hand, the cells with high degree of differentiation have remaining structural and functional properties of internal organs and thus have a good prognosis. Therefore, the composition comprising an aggregate containing fibroblasts with high degree of differentiation can be used preferably to repair a defect in soft tissue.

A preferred mode of the composition of the present invention is a composition for repairing a defect of soft tissue of the skin, which is used for cosmetics such as removing wrinkles or flaps of the skin, and which can also be used as a pharmaceutical composition for recovering soft tissue of the skin lost from some disease. Though the preferred usage of this composition is an injectable solution, it may be used for other applications.

Moreover, the preferred mode of the composition of the present invention is a composition for repairing a defect in soft tissue of gingival, which can be used for the treatment of the gingival recession etc. caused by aging, wound on gingival or gum, surgical treatment associated with cutting gingival, etc. Though the preferred usage of this composition is an injectable solution, it may be used for other applications.

The third aspect of the present invention relates to a method of producing an injectable solution. Specifically, the method of producing an injectable solution comprises the step of culturing in an autologous serum medium or a serum-free medium with vitamin C added, the step of culturing autologous fibroblasts by a culture method selected from among rotary culture, shaking culture and slant culture so as to form a fibrous aggregate by an extracellular matrix containing collagen secreted by the autologous fibroblasts, and the step of exfoliating the cultured cells as an aggregate, wherein after the aggregate obtained in the step of collecting is inserted into a syringe through a first injection needle, the first injection needle is removed from the syringe and a second injection needle with a smaller inner diameter than that of the first injection needle is attached to the syringe. In this way, an aggregate is once sucked up with an injection needle with a larger inner diameter, which is later replaced by an injection needle with a smaller inner diameter. Consequently, the aggregate can be formed into an appropriate size during treatment, physical damage to cells can be minimized, and in addition the situation that the aggregate clots in the injection needle can be prevented effectively.

The aggregate comprising autologous fibroblasts of the present invention is formed of fibroblasts combined with one another through an extracellular matrix containing collagen. If an aggregate is too large, it cannot be injected with an injection needle, and if it is too small, it does not remain in the affected area. From such a viewpoint, the aggregate contained in an injectable solution is, in its form and size, preferably fibrous, can be suctioned with a 18-20G (gauge) injection needle, and can be injected with a 24-30G injection needle. The aggregate of this size can be obtained by adjusting the rate of cells to culture area of a culture vessel, by adjusting the concentration of the separation liquid for exfoliating cells, or by leaving to stand exfoliated and collected cells. The rate of cells to culture area of a culture vessel is 70-100% confluent, for example. It may be 80-90% confluent or 90-95% confluent. The concentration of the separation liquid for exfoliating cells can be adjusted appropriately depending on the separation liquid used. When using a separation liquid containing trypsin, the trypsin concentration in the separation liquid is 0.1-2%, for example. If the cellular aggregate is small when the cells are exfoliated, the exfoliated cells are collected in a well-known vessel and are left to stand. The time of leaving to stand is 30 seconds to 10 minutes, for example. It may be 1-3 minutes or 3-5 minutes. It may be adjusted appropriately depending on the size of the exfoliated cells. By leaving to stand and precipitating the cells, the exfoliated cells are combined with one another again, resulting in enlarging the size of an aggregate. An aggregate can be obtained by loosening by tapping etc. the precipitated cells.

Hereinafter, the present invention will be described in detail with reference to the examples. However, the present invention is not limited to the examples below. That is, the present invention can be modified appropriately within the scope apparent to those skilled in the art, and such modifications are also included in the present invention.

### Example 1

### Example 1

### Preparation of Aggregate of Autologous Fibroblasts

Culture of the fibroblasts separated from skin tissue was carried out according to a well-known method (R. I. Freshney, Culture of Animal Cells: A MANUAL OF BASIC TECHNIQUE (Wiley-Liss, Inc. 2000)). Specifically, the fibroblasts separated from skin tissue were being subcultured, subsequently followed by expansion culture, to be end up to a few T-225 flasks in the end. Vitamin C in the range of 0.01-1 mM was added to the culture medium three days before the end of culture (scheduled day of injection). Furthermore, the culture medium was replaced with a 2-5% self-serum addition culture medium with no vitamin C added. The cultured fibroblasts were exfoliated from a culture vessel with a dispersion liquid with no EDTA solution or enzyme, and the cells were separated from the dispersion liquid by centrifugation. Then, washing in a physiological salt solution were performed more than three times.

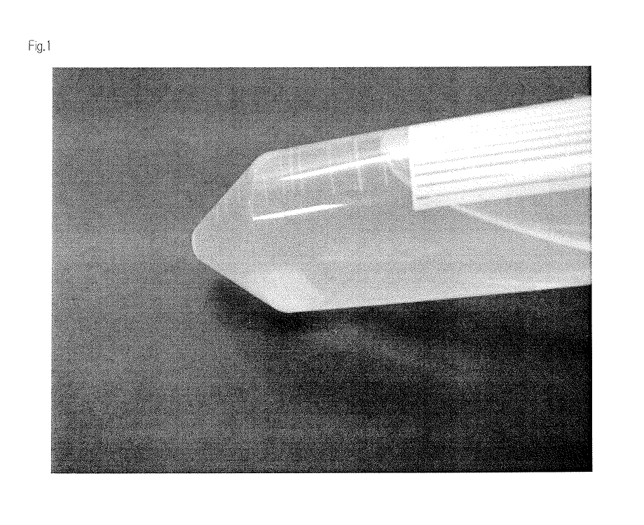

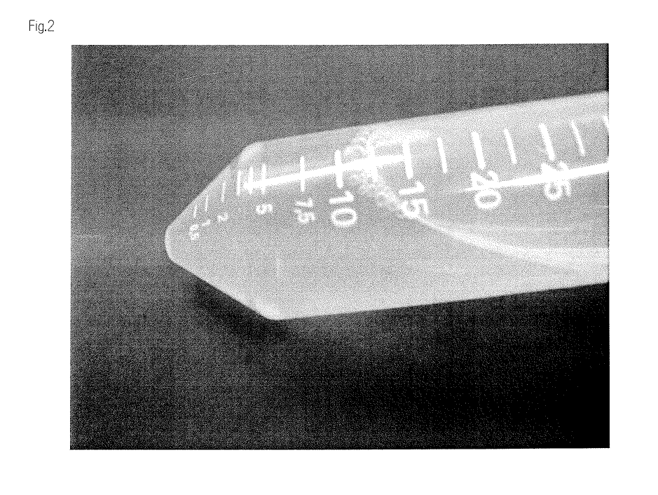

The dispersion liquid obtained in this way is shown in FIG. 1. FIG. 1 shows a photograph replacing a drawing which shows a liquid medicine containing fibroblasts obtained from Example 1. On the other hand, FIG. 2 shows a dispersion liquid where fibroblasts are exfoliated with an EDTA solution or enzymes with no vitamin C added to the culture medium. FIG. 2 shows a photograph replacing a drawing which shows a liquid medicine containing fibroblasts obtained by a conventional method.

When FIG. 1 is compared with FIG. 2, it is understood that the conventional method is superior as an appearance of the injection solution used for cosmetics. And, as can be seen from FIG. 1, fibroblasts form a large number of aggregates according to the above method. On the other hand, as can be seen from FIG. 2, aggregates are not formed by the conventional method.

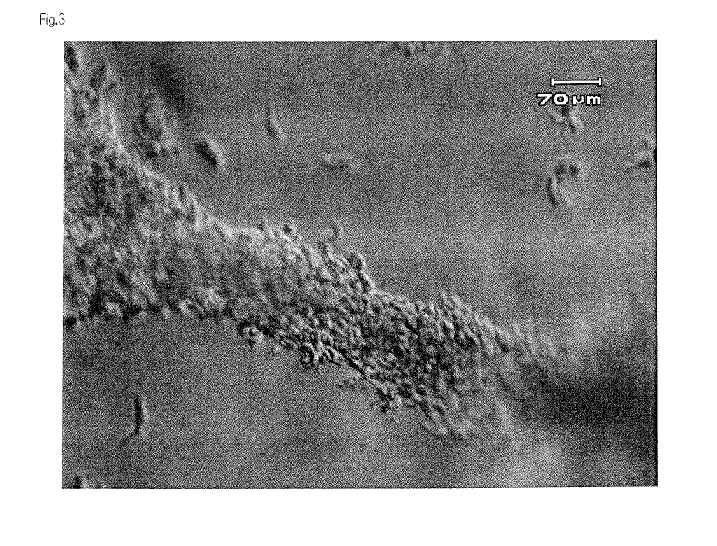

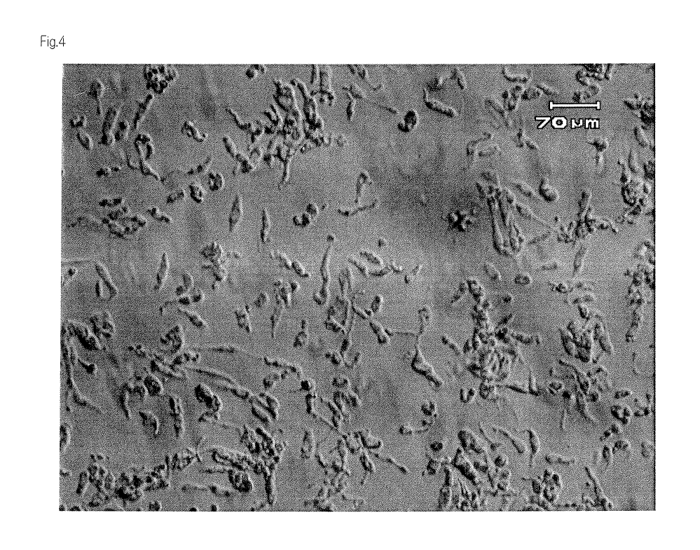

The cellular aggregate was dispersed with a pipette from the liquid medicine containing the fibroblasts obtained from Example 1, suspended in a physiological salt solution and micrographed. The result is shown in FIG. 3. FIG. 3 shows a photograph replacing a drawing which shows a situation where the aggregate obtained from Example 1 was dispersed. On the other hand, the cellular aggregate in the separation liquid where fibroblasts are exfoliated with an EDTA solution or enzymes with no vitamin C added to the culture medium was dispersed with a pipette, suspended in a physiological salt solution and micrographed. The result is shown in FIG. 4. FIG. 4 shows a photograph replacing a drawing which shows a situation where the cellular aggregate of fibroblasts was dispersed.

As can be seen from FIG. 4, if the fibroblasts were obtained by the conventional method, the cells are dispersed. On the other hand, as can be seen from FIG. 3, according to the present invention, fibroblasts are combined with collagen fibers, and a band-like cellular aggregate can be obtained. Particularly, as the cellular aggregate becomes band-lie (fibrous) this way, it can be sucked and injected with an injection needle. However, there were fewer aggregates which became band-like (fibrous).

### Example 2

### Example 2

### Effect to Collagen Production by Adding Vitamin C

The collagen concentration contained in the culture supernatant of the fibroblasts cultured in the culture medium with vitamin C added of Example 1 and the culture supernatant with no vitamin C added by the human collagen ELISA quantitative kit (AC Biotechnologies). The result is shown in Table 1.

TABLE 1

| | |
|---|---|
| Added | 11.3 |
| Not added | 1.05 |

As can be seen from Table 1, by adding vitamin C to a culture medium, the capacity to produce collagen is increased dramatically. This means that adding vitamin C to a culture medium helps fibroblasts to form an aggregate.

### Example 3

### Example 3

### Verification of the Effect of the Composition of the Present Invention

Cells were separated from the pieces of skin tissue extracted from four volunteers by the outgrowth method, which were cultured in an autologous serum addition culture medium by the method shown in Example 1. The liquid containing aggregates was suspended in a high calorie infusion solution and stored at 4 degrees C. The fibroblasts in a suspended state had formed aggregates as shown in FIG. 1. Before injection, the cells were separated from the infusion solution by centrifugation, and an appropriate amount of physiological salt solution was added to the cells, which was agitated lightly into a suspended state. Each aggregate was sucked into a syringe with an 18G injection needle, replaced with a 26G or 27G injection needle, and injected into the desired local area.

After injection, doctors and cosmetics specialists judged four volunteers' effects. When the judgments by these specialists were not obtained, self-valuation was performed. The result is shown in Table 2.

TABLE 2

| | | 3 | 6 |
|---|---|---|---|
| | 1 week later | months later | months later |
| Effect of | 4/4 | 4/4 | 4/4 |
| disappearance of wrinkles | | | |
| Effect of | 3/3 | 3/3 | 3/3 |
| disappearance of dark circles | | | |
| under eyes | | | |

Note:
In the table, the denominators indicate the number of people who received treatment and the numerators indicate the number of people who benefited from treatment.

Although in all volunteers the swellings went down on the next day when the injectable solution was injected, elevations were observed in the injected portions. Several days later, tensions appeared also in the circumference of the injected portions, the depressed portions were restored, and wrinkles disappeared. Furthermore, dark circles under eyes also disappeared, which showed medical and cosmetics effects. These effects continued at least for one and half a year in all volunteers.

### Example 4

### Example 4

### Examination of Effect on the Cell Culture by a High Calorie Injectable Solution

The fibroblasts cultured by a common method were exfoliated from a flask, and it was confirmed that the percentage of the living cells had been 100% soon after that. Then, suspended matter of 2×10⁷ fibroblasts suspended in 25 ml of physiological salt solution in a 50 ml disposal centrifugation pipe and suspended matter of the same number of fibroblasts suspended in 25 ml of high calorie injectable solution (amino freed etc.) were prepared. After conveying these in a cooling state with the refrigerant for 2 hours, the number of the living cells was measured. Then, one fifth of the cells in each suspended matter were cultured with T-225 flasks, respectively.

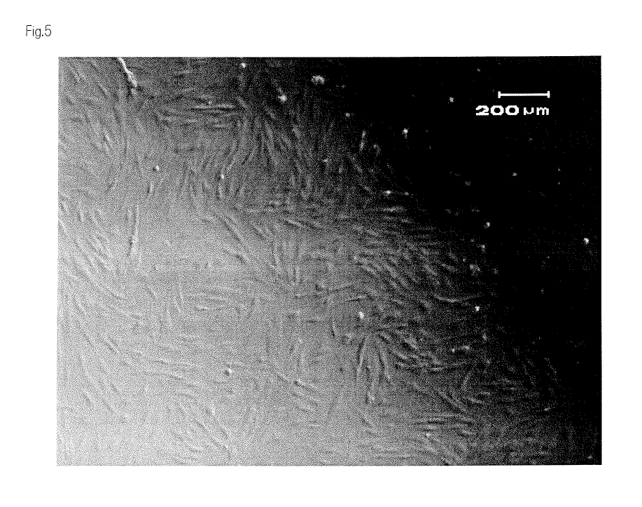

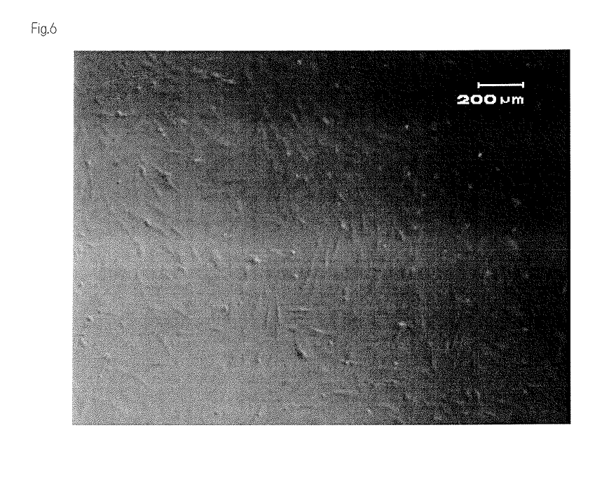

The number of the living cells after trypan blue dyeing and the number of the dead cells were measured with a counting chamber. The rate of the living cells is shown in Table 3. Moreover, the cells after flask culture for 24 hours were photographed. FIG. 5 shows a photograph replacing a drawing which shows a situation where cells are suspended in a high calorie injection solution. On the other hand, FIG. 6 shows a photograph replacing a drawing which shows a situation where cells are suspended in a physiological salt solution.

TABLE 3

| Suspension liquid | Rate of living cells |
|---|---|
| High calorie injectable solution | 95% |
| Physiological salt solution | 85% |

As can be seen from Table 3, the rate of living cells is 10% higher in suspended matter in a high calorie injection than that in physiological salt solution. Moreover, as can be seen from FIG. 5 and FIG. 6, it is apparent that the cells suspended in a high calorie injection solution also have better growth ability.

### Example 5

### Example 5

### Analysis of Protein Component of Cellular Aggregate

The cellular aggregate produced in Example 1 was suspended in a physiological phosphate buffer (PBS). And, in order to further disperse the cells, dispase I (Godo Shusei) was prepared to 1,000 PU/ml with final concentration, and was processed at 37 degrees C. for 1 hour. Then, the cells and supernatants were separated by centrifugation, and the proteins with more than 3000 molecular weight in the supernatants were condensed with a centrifugal concentrator (Millipore). The protein components were separated as a spot by two-dimensional cataphoresis, and the spot was cut out from gel, and the proteins were identified with a mass spectrometer after trypsin digestion.

As a result, extra-cellular matrixes such as type I collagen, type VI collagen, fibronectin, vimentin, and microfibril related glycoprotein were detected.

### Example 6

### Example 6

### Preparation Method of a Fibrous Cellular Aggregate

In order to further increase the rate of fibrous cellular aggregates compared to Example 1, the fibroblasts separated from the pieces of skin tissue were cultured by the following two methods:

(1) The fibroblasts cultured in two T-225 flasks until they became 100% confluent respectively were disseminated to a roller bottle (surface area of 850 cm²), and rotary culture was carried out at a rate of 0.2-2 revolutions per minute. The optimal condition was 0.4-0.5 revolutions per minute.

(2) Shaking culture of the cells disseminated to the T-225 flasks with a shaking machine (5-50 times per minute) which performs horizontal, vertical, and gyratory motions. The optimal condition was vertical shaking (10-20 times per minute) culture. The composition of culture media and the culture environment were same as those in Example 1.

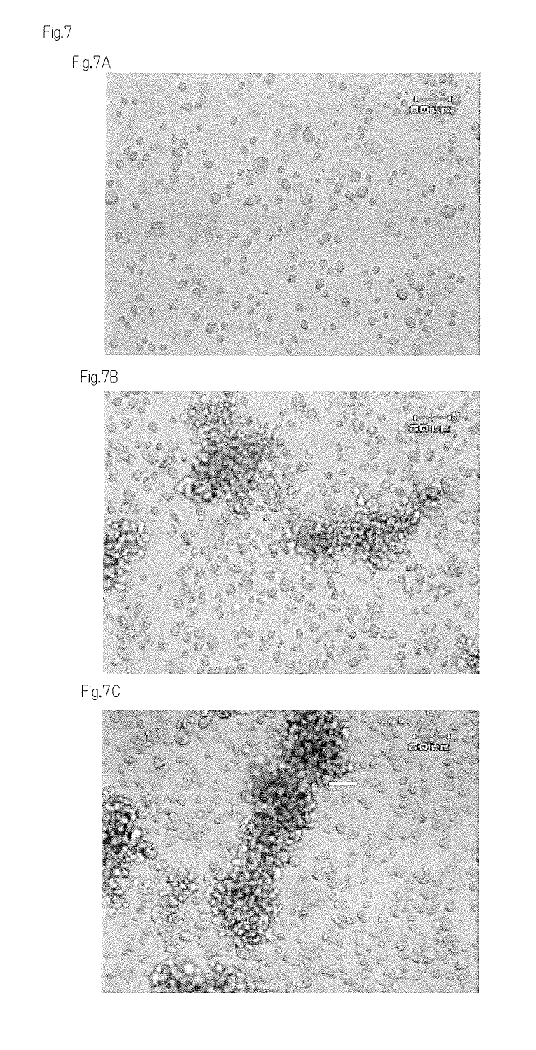

The fibroblasts were cultured until they became 100% confluent, and the cells were dispersed with a cell dispersion liquid and were collected. As a cell dispersion liquid, a type of dispersion liquid which does not use trypsin (no animal derived ingredients) (e.g. TrypLE Select, TrypLE Express, etc. (Invitrogen)) was used. When a dispersion liquid of the same concentration was added and the cells were incubated and dispersed on the same conditions, there were differences in the presence of absence of cellular aggregates between stationary culture and rotary culture. The result was shown in FIG. 7. FIG. 7A shows the dispersed and collected fibroblasts which were cultured in a flask by stationary culture and FIGS. 7B and 7C show the dispersed and collected fibroblasts which were cultured by rotary culture. As shown in FIG. 7, the cells by stationary culture did not form aggregates but were in a usual dispersed state. On the other hand, the cells by rotary culture formed fibrous cellular aggregates. The cellular aggregates are large enough to be injected into a 24-26G (gauge) injection needle and the rate or size of the cellular aggregates can be adjusted depending on culture time and the concentration of a separation liquid. Moreover, the rated or size of the fibrous cellular aggregates shown in FIG. 7B and FIG. 7C can be adjusted also by being left to stand and precipitated after collection.

### Example 7

### Example 7

### Characteristics of Fibrous Cellular Aggregates 1: Comparison of Quantity of Collagen

The quantity of collagen attached to the cellular aggregates by rotary culture and the isolated cells by stationary culture each obtained from the same culture area. More specifically, each cell pellet was washed with a physiological salt solution several times, and pepsine (SIGMA) was added to the cell pellets so that the final concentration might be 0.01-0.5 mg/mL, which had been shaken at 4 degrees C. overnight to extract collagen, and the quantity of collagen in the supernatants was measured using the human collagen Type I ELISA kit (AC Biotechnologies). The result is shown in Table 4. As shown in Table 4, the cellular aggregates by rotary culture were shown to include about five times as much collagen as the isolated cells by stationary culture. If the cellular aggregates obtained by rotary culture are converted into the quantity of collagen contained in the cellular aggregates per culture area of 100 cm², it would be equivalent to more than 2 μg.

TABLE 4

| | Quantity of collagen | Rate |
|---|---|---|
| Flask stationary culture | 0.37 μg | 1 |
| Rotary culture | 1.77 μg | 4.8 |

### Example 8

### Example 8

### Characteristics of Fibrous Cellular Aggregates 2—Growth Ability of Cellular Aggregates

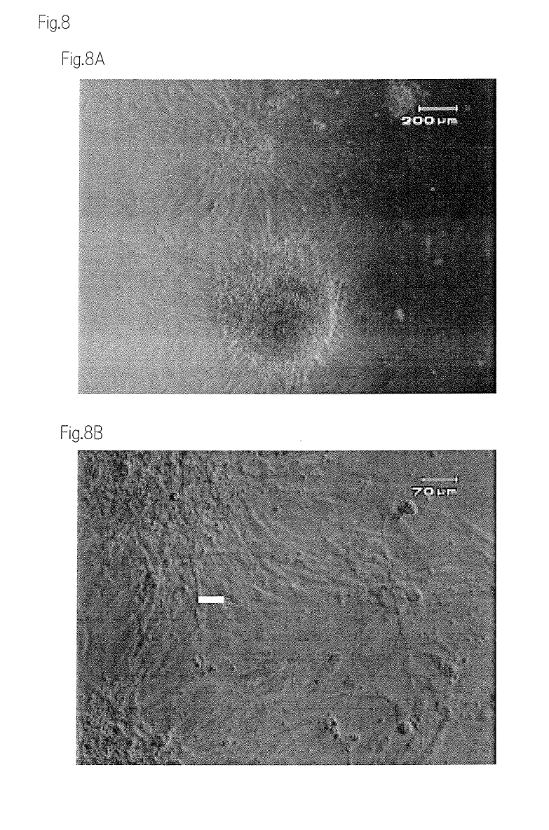

Whether the prepared cellular aggregates exist or not was confirmed by the following method. That is, the cells by rotary culture using a roller bottle were exfoliated with a dispersion liquid, and then the dispersion liquid was removed by centrifugation to obtain cell pellets. The cells were put into a centrifugation pipe, and the pellets were dissolved by pipetting and were left at 4 degrees C. for 8 hours. Then, the cell pellets were again dissolved by pipetting and were left to stand for 10 minutes. As the cellular aggregates precipitated and the isolated cells were suspended, the precipitated cellular aggregates were collected and cultured in a flask. The cellular aggregates 24 hours after the start of culture were attached to the flask (FIG. 8A), and the attached cells began to grow 48 hours later (FIG. 8B). Thus, it turned out that the aggregated cells can grow even after a certain period of time at 4 degrees C.

In this way, the aggregated cells can grow again by the start of culture even after a certain period of time at 4 degrees C., which shows that there is a high possibility of cell growth in vivo as well.

### Example 9

### Example 9

### Spot Improvement Effect

Fibroblasts were extracted from the volunteer from whom the consent of participation to clinical testing was obtained, and were cultured. Then, the cellular aggregates were adjusted, and the adequate amount (a total of 5×10⁶ cells) was injected into the circumference of the 2 cm×1 cm spot (liver spot) in the right temple of the face twice at two-week intervals. As a result, the spot started thinner from one month later, and it disappeared completely (only the trace remained) half a year later, and the disappeared state has been maintained for more than one and a half years thereafter. During that time, the volunteer did not do anything particular and used common cosmetics.

### Example 10

### Example 10

### Growth Ability of Gingival-Derived Fibroblasts

### Separation Method of Gingival-Derived Fibroblasts

An extracted tooth was obtained from a visiting patient with the consent of the use for research purposes. After it was washed well in a physiological salt solution where antibiotics (500 U/ml penicillin and 500 μg/ml streptomycin) were added, the gingival added to the circumference of the extracted tooth was separated with a scalpel, and the piece of gingival tissue was chopped. The piece of gingival tissue was attached to a 6-well tissue culture plate and was left to stand for five minutes. And the culture medium with antibiotics and 10% autologous serum were added in as small amounts as possible to the fixed piece of gingival tissue so that the piece of gingival tissue could soak in the culture medium, and the piece of gingival tissue was cultured in a CO₂ incubator at 37 degrees C. The above culture solution was added to the fixed (attached) piece of tissue and cultured for about two weeks. At the point when the cells migrated from the piece of tissue and grew to be locally confluent, subculture was started. The subculture was carried out as described below. The cells were washed in a physiological salt solution and were suspended by a separation liquid. Then, autologous serum was added to inactivate enzymes, or a dispersion solution was diluted with a physiological salt solution, and the cells were collected by centrifugation. These cells were suspended in the above culture medium, disseminated to and cultured in a T-75 flask. After that expansion culture was carried out until the desired number of cells was reached.

### Growth Ability of Gingival-Derived Fibroblasts

Dissemination of skin-derived fibroblasts and dissemination of gingival-derived fibroblasts were carried out in 96-well plates at a rate of 5×10³ cells/well, respectively. The cells were cultured for 1, 2, and 3 days from the day after culture, and the cell growth ability was compared by the color (absorbance of 450 nm) of WST-1 (Takara Bio) reagents. If the absorbance is higher, the viability of cells is higher, and the number of cells is also increased proportionately. The result was shown in Table 5. As shown in Table 5, the growth ability of gingival-derived fibroblasts was higher than that of skin-derived fibroblasts.

TABLE 5

| | Absorbance (450 nm) | | |
|---|---|---|---|
| Fibroblasts | 1 day later | 2 days later | 3 days later |
| Skin-derived | 1.24 | 1.33 | 1.89 |
| Gingival-derived | 1.83 | 2.80 | 3.31 |

### Example 11

### Example 11

### Preparation Method of Fibrous Aggregates of Gingival-Derived Fibroblasts

According to the above-mentioned preparation method of fibrous cellular aggregates, gingival-derived fibroblasts were disseminated in a roller bottle and cultured by rotary culture, or were disseminated in a flask and cultured by shaking culture. The cells were made 100% confluent, and exfoliated with a cell dispersion liquid and dispersed. Like skin-derived fibroblasts, the cells by rotary culture formed aggregates. Like Example 7 (Table 4), the quantity of collagen in the cell pellets obtained by rotary culture and stationery culture in a flask under the same culture conditions was measured. The result was shown in Table 6. As shown in Table 6, the cellular aggregates obtained by rotary culture were shown to include about sixteen times as much collagen as the cells obtained from stationary culture. If the cellular aggregates obtained by rotary culture are converted into the quantity of collagen contained in the cellular aggregates per cultivation area of 100 cm², it would be equivalent to more than 200 μg. When comparing the cellular aggregates obtained by rotary culture of skin-derived fibroblasts in Example 7 and the cellular aggregates obtained by rotary culture of gingival-derived fibroblasts in Example 11, it tuned out that the number of cellular aggregates obtained from-gingival derived fibroblasts are 100 times as many as that of the cellular aggregates obtained from skin-derived fibroblasts.

TABLE 6

| | Quantity of collagen | Rate |
|---|---|---|
| Flask stationary culture | 10.6 μg | 1 |
| Rotary culture | 168 μg | 15.8 |

### Example 12

### Example 12

### Characteristics of Cellular Aggregates 1: Examination of ALP Activity

The degree of differentiation of gingival-derived fibroblasts was examined with ALP as an index. After the above cells and cellular aggregates were washed with PBS (−), they were fixed for 10 minutes with methanol and washed with PBS (−) several times. ALP dyeing was performed as described below according to the method of Harlow and Lane (Antibodies: A Laboratory Manual, Cold Spring Harbor Laboratory, Cold Spring Harbor, N.Y. (1988)).

That is, a reaction solution where 0.1M Tris-HCl buffer solution (pH 9.5) containing 0.05M MgCl₂ and 0.15 mg/ml 5-bromo-4-chrolo-3-indolyl and 0.3 mg/ml nitroblue tetrazolium chloride (both Invitrogen) were dissolved was added to the cells and the cellular aggregates and reacted for two hours at room temperature in the dark. As a result, they were not dyed in two hours, but were dyed blue for a while after they were left overnight, and thus it turned out that they had expressed almost no ALP (no ALP activity).

From the result in Example 12, it tuned out that as the fibroblasts obtained by the present invention had expressed almost no ALP activity, they were differentiated fibroblasts. It is known that the cells with low degree of differentiation are quick to grow after transfer, are easy to develop into canceration and have a poor prognosis. As the fibroblasts obtained by the present invention have high degree of differentiation and thus are regarded as not easy to develop into canceration, they can be used preferably for repairing a defect in gingival soft tissue.

### Example 13

### Example 13

### Characteristics of Cellular Aggregates 2: Measurement of GGT Activity

Periodontal bacteria exist in a periodontal pocket and express GGT causing inflammatory responses, and thus there is a possibility that inflammatory cells (T-cell or macrophage) which express GGT or periodontal bacteria are mixed in the separated fibroblasts. Therefore, GGT activity was measured in order to confirm that the separated fibroblasts had not expressed GGT.

GGT (γ-glutamyl transpeptidase) activity in the culture supernatant cultured in the above example was measured using the kit by AC Biotechnologies (GGT activity measurement kit (PN: AD101)). This kit is a measuring method based on the recommended method (JSCC), and GGT prepares L-γ-glutamylglycylglycine and 5-amino-2-nitrobenzoic acid under the presence of donor substrate glycylglycine.

With regard to the prepared 5-amino-2-nitrobenzoic acid, the increase in the absorbance (410 nm) per unit time was measured to obtain the activity value. The result was shown in Table 7. As shown in Table7, there was no increase in GGT activity depending on cultivation time, and both of the skin and the gingival fibroblasts had the same activity. Therefore, GGT activity of the fibroblasts obtained by the present invention is negative, and by culturing the cells by rotary culture, the cellular aggregates having the effect of raising gingival can be prepared.

TABLE 7

| Fibroblasts | 1 day later | 2 days later | 3 days later |
|---|---|---|---|
| Skin-derived | 11.8 | 12.0 | 13.6 |
| Gingival-derived | 13.8 | 13.4 | 13.6 |

### INDUSTRIAL APPLICABILITY

The present invention may be used effectively in the cosmetic industry, beauty industry, etc. as cosmetics, for example. Furthermore, the present invention also may be used effectively in the medical field such as dentistry.

1. A method of producing an aggregate of autologous fibroblasts comprising:
a step of culturing autologous fibroblasts which attach to a vessel for culture in a culture medium with vitamin C added;
a step of culturing autologous fibroblasts by rotary culture, keeping the autologous fibroblasts attached to the vessel for culture, the rotary culture being carried out at a rate of 0.2-2 revolutions per minute, so as to form a fibrous aggregate by an extracellular matrix containing collagen secreted by the autologous fibroblasts; and
a step of removing the rotary cultured cells from the vessel for culture.

2. The method as claimed in claim 1,
wherein the step of removing cells from the vessel for culture is a step of removing cells with a separation liquid comprising trypsin.

3. The method as claimed in claim 1,
wherein the step of culturing in a culture medium is a step of culturing in an autologous serum medium or in a serum-free medium.

4. The method as claimed in claim 1,
wherein the autologous fibroblasts are either skin-derived fibroblasts or gingival-derived fibroblasts.

5. The method as claimed in claim 1,
wherein the step of culturing is a step of culturing by rotary culture, the rotary culture being carried out at a rate of 0.1-2 revolutions per minute.

6. The method as claimed in claim 1,
wherein the step of culturing is a step of culturing by shaking culture, the shaking culture being any of vertical motion culture, horizontal motion culture or gyratory culture, and the shaking being carried out at a rate of 5-50 times per minute.

7. The method as claimed in claim 1,
wherein the step of culturing is a step of culturing by rotary culture,
the step of culturing by rotary culture comprising following steps in this order:
a step of culturing cells at a rate of 0.1-0.5 revolutions per minute;
a step of adding vitamin C to a culture medium so that the density of the vitamin C becomes 0.01-1 mM and culturing cells at a rate of 1.5-2 revolutions per minute; and
a step of culturing cells at a rate of 0.5-1.5 revolutions per minute.

8. The method as claimed in claim 1,
wherein the step of culturing is a step of culturing by shaking culture, and
wherein the step of culturing by shaking culture comprising following steps in this order:
a step of culturing cells at a rate of 5-10 times per minute;
a step of adding vitamin C to a culture medium so that the density of the vitamin C becomes 0.01-1 mM and culturing cells at a rate of 20-30 times per minute; and
a step of culturing cells at a rate of 10-20 times per minute.

9. The aggregate produced by the method as claimed in claim 1.

10. A composition for repairing a defect in soft tissue comprising an aggregate and a pharmaceutically acceptable liquid medicine,
wherein the aggregate is produced by a method of producing an aggregate of autologous fibroblasts comprising: a step of culturing in an autologous serum medium or in a serum-free medium with vitamin C added; and a step of culturing autologous fibroblasts by a culture method selected from among rotary culture, shaking culture and slant culture so as to form a fibrous aggregate by an extracellular matrix containing collagen secreted by the autologous fibroblasts, and
wherein the pharmaceutically acceptable liquid medicine is a liquid medicine containing carbohydrates, electrolytes and amino acids.

11. The composition as claimed in claim 10,
wherein the aggregate is an aggregate containing differentiated fibroblasts prepared from gingival-derived fibroblasts.

12. A method of producing an injectable solution comprising:
a step of culturing autologous fibroblasts which attach to a vessel for culture in an autologous serum medium or in a serum-free medium with vitamin C added;
a step of culturing autologous fibroblasts by rotary culture, keeping the autologous fibroblasts attached to the vessel for culture, the rotary culture being carried out at a rate of 0.2-2 revolutions per minute, so as to form a fibrous aggregate by an extracellular matrix containing collagen secreted by the autologous fibroblasts;
a step of removing the rotary cultured cells from the vessel for culture;
a step of inserting the aggregate obtained in the step of removing into a syringe through a first injection needle;
a step of removing the first injection needle; and
a step for attaching a second injection needle to the syringe, wherein the second injection needle has a smaller inner diameter than that of the first injection needle.

13. The method of producing an aggregate of autologous fibroblasts as claimed in claim 1, wherein the rotary culture is carried out at a rate of 0.4-0.5 revolutions per minute.

14. The method of producing an injectable solution as claimed in claim 12, wherein the rotary culture is carried out at a rate of 0.4-0.5 revolutions per minute.
